(19) United States
(12) Patent Application Publication
(10) Pub. No.: US 2008/0102453 A1
(43) Pub. Date: May 1, 2008

(54) Methods and systems and analysis of CGH data

(76) Inventors: Jayati Ghosh, Sunnyvale, CA (US); Bo U. Curry, Redwood City, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/591,222

(22) Filed: Oct. 31, 2006

(51) Int. Cl. C12Q 1/68; G06F 19/00

(52) U.S. Cl. 435/6; 702/20

(57) ABSTRACT

Methods, systems and computer readable media for analysis of comparative genomic hybridization data analysis, including creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions; identifying a peak corresponding to regions of normal copy number in the centralization curve; centralizing the log ratio data so that the peak corresponding to regions of normal copy number is centered at a log ratio value of zero; calculating a mathematical measurement that is a function of the width of the peak corresponding to regions of normal copy number; calculating a tolerance value as a function of the mathematical measurement; and outputting the tolerance value. Methods, systems and computer readable media are provided to create a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions; identify peaks in the centralization curve; assign copy numbers to the identified peaks; plot expected ratios, based on the assigned copy numbers, of the peaks versus observed ratios of the peaks calculated from the log ratio data values; conclude that the assigned copy numbers are correct if the plot of the expected ratios versus the observed ratios is substantially linear; and output at least one of the plot of expected ratios versus observed ratios, and a conclusion as to whether the plot is substantially linear.

### BACKGROUND OF THE INVENTION

Many genomic and genetic studies are directed to the identification of differences in gene dosage or expression among cell populations for the study and detection of disease. For example, many malignancies involve the gain or loss of DNA sequences (alterations in copy number), sometimes entire chromosomes, that may result in activation of oncogenes or inactivation of tumor suppressor genes. Identification of the genetic events leading to neoplastic transformation and subsequent progression can facilitate efforts to define the biological basis for disease, improve prognostication of therapeutic response, and permit earlier tumor detection. In addition, perinatal genetic problems frequently result from loss or gain of chromosome segments such as trisomy 21 or the micro deletion syndromes. Trisomy of chromosome 13 results in Patau syndrome. Abnormal numbers of sex chromosomes result in various developmental disorders. Thus, methods of prenatal detection of such abnormalities can be helpful in early diagnosis of disease.

Comparative genomic hybridization (CGH) is a technique that is used to evaluate variations in genomic copy number in cells. In one implementation of CGH, genomic DNA is isolated from normal reference cells, as well as from test cells (e.g., tumor cells). The two nucleic acids are differentially labeled and then simultaneously hybridized to an array of oligonucleotide probes. Array CGH (aCGH) offers benefits over earlier methods, including a higher resolution, as defined by the ability of the assay to localize chromosomal alterations to specific areas of the genome. For further detailed description regarding aCGH technology, the reader is referred to co-pending application Ser. No. 10/744,495 filed Dec. 22, 2003 and titled “Comparative Genomic Hybridization Assays Using Immobilized Oligonucleotide Features and Compositions for Practicing the Samet”; application Ser. No. 11/545,962 filed Oct. 10, 2006 and titled “Analyzing CGH Data to Identify Aberrations”; application Ser. No. 11/338,515, filed Jan. 24, 2006 and titled “Method and System for Determining a Zero Point for Array-Based Comparative Genomic Hybridization Data”; and application Ser. No. 10/953,958, filed Sep. 29, 2004, published on Apr. 20, 2006 as U.S. Patent Application Publication No. 2006/0084067, and titled “Method and System for Analysis of Array-Based, Comparative-Hybridization Data”, each of which is incorporated herein, in its entirety, by reference thereto.

aCGH assays measure the differences in copy number between a test sample and a reference sample. For example, two genomic samples (a test sample and a reference sample) can be labeled with two different dyes and hybridized together to a single microarray to perform these measurements. Alternatively, the two different samples can be hybridized to separate arrays and then measurements can be compared between the arrays. In any case, the log ratio of the test sample signal to corresponding signal from the reference sample for the same probe is measured, and this is typically done for each probe that both test and reference samples have been hybridized to (note, the “same” probe can be probes on two different arrays as long as it codes for the same sequence). The signals compared between the test and reference samples (e.g., two channels measured from the same array, or two separate channels from two different arrays) are typically normalized so that the median log ratio of the signals from the two samples is zero. However, what is really preferred when attempting to identify aberrations is to report an average log ratio of zero for those probes in chromosomal regions having the “normal” ploidy of the species (e.g., which is typically the autosomal ploidy of the reference sample. In cases where the test sample is highly aneuploid, normalization of the median log ratio signal (log ratio test/reference signals) will not, in general, result in a reported log ratio of zero for regions of normal copy number. Consequently, an additional normalization step is required to explicitly set the log ratio of normal regions to zero. This additional step has been termed “centralization”, to distinguish it from the earlier performed channel normalization technique that is naïve with regard to chromosome positions from which signals are derived. This centralization technique is described in application Ser. No. 11/338,515 which was incorporated by reference above.

Even when the data have been properly centralized, some chromosomal regions, or even entire chromosomes can have average log ratios reported that are slightly different from zero. While these non-zero reported ratios for regions that are not aberrant may result from statistically significant differences in the concentrations of labeled targets (between test sample and reference sample) from these chromosomes, or from other sources of noise in the assay, they are generally not indicative of real copy number differences between the samples. Rather, they are thought to result from accidental variations introduced during sample isolation, amplification, labeling, etc. In view of this phenomenon, there is a continuing need for improved methods and systems for more accurately reporting CGH data comparisons, such that such accidental non-zero ratios are not reported as if they were potentially true copy number variations, regardless of the statistical significance of these accidental non-zero ratios.

### SUMMARY OF THE INVENTION

Methods, systems and computer readable media are provided for comparative genomic hybridization data analysis, to include the steps of: inputting log ratio data values for DNA copy numbers of a genome of a test sample relative to a genome of a reference sample, wherein the reference sample has a known ploidy, and the test sample has a same copy number as the reference sample in normal, non-aberrant genomic regions; creating a centralization curve from the log ratio data values; identifying a peak corresponding to regions of normal copy number in the centralization curve; centralizing the log ratio data so that the peak corresponding to regions of normal copy number is centered at a log ratio value of zero; calculating a mathematical measurement that is a function of the width of the peak corresponding to regions of normal copy number; calculating a tolerance value as a function of the mathematical measurement; and outputting the tolerance value.

In at least one embodiment, an aberration calling algorithm is run on the log ratio data values, including the tolerance value as an input to the aberration calling algorithm for setting upper and lower threshold values; and genomic regions represented by portions of the log ratio data having an average log ratio that is non-zero, but is within the upper and lower thresholds are called as normal regions.

In at least one embodiment, the peak corresponding to regions of normal copy number is the most prominent peak in the centralization curve.

In at least one embodiment, the peak corresponding to regions of normal copy number is selected heuristically.

In at least one embodiment, the calculation of a mathematical measurement that is a function of the width of the peak corresponding to regions of normal copy number comprises fitting N Gaussian curves to N identified peaks, wherein N is a positive integer and the N peaks include the peak corresponding to regions of normal copy number; and wherein each of the Gaussian curves is defined to have the same variance.

In at least one embodiment, the centralization curve is created by a histogram.

In at least one embodiment, the centralization curve is created by: (a) plotting the log ratio data against an initial assumed location of an axis indicating a log ratio of zero; (b) running an aberration calling algorithm on the data; (c) tallying the fraction of the data points called in non-aberrant regions; (d) storing the location of the axis and the fraction of non-aberrant data points as a data pair; (e) incrementing the position of the axis indicating a log ratio of zero by a predetermined incremental value; (f) repeating steps (b)-(e) until the axis has been incrementally moved from the initial location for log ratio of zero to both predetermined positive and negative end locations; and (g) plotting the data pairs, with the axis position for zero log ratio values plotted along one axis and corresponding fraction values plotted along a second axis.

Methods, systems and computer readable media are provided for comparative genomic hybridization data analysis, to include the steps of: creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions; identifying peaks in the centralization curve; assigning copy numbers to the identified peaks; plotting expected ratios, based on the assigned copy numbers, of the peaks versus observed ratios of the peaks calculated from the log ratio data values; concluding that the assigned copy numbers are correct if the plot of the expected ratios versus the observed ratios is substantially linear and the substantially linear plot is within a range of expected slope values; and outputting at least one of the plot of expected ratios versus observed ratios, and a conclusion as to whether the plot is substantially linear.

In at least one embodiment, if the plot is not substantially linear, the following further steps are carried out: reassigning a different copy number to at least one of the identified peaks to establish a new assignment of copy numbers;

repeating the plotting of expected ratios versus observed ratios, using the new assignment of copy numbers; determining whether the plot from the repeating step is substantially linear; and iterating the reassigning, repeating and determining steps until it is determined that the plot is substantially linear.

In at least one embodiment, the expected ratios are calculated as the quantity k/2-1, where k is the assigned copy number, and the slope of the plot is calculated, wherein the slope identifies the fraction of the test sample that is aberrant.

In at least one embodiment, the value of the fraction of the test sample that is aberrant is outputted.

In at least one embodiment, multiple peak groupings indicative of a multi-clonal test sample are identified in the centralization curve, wherein the assignment of copy numbers to the identified peaks comprises assigning the same copy number to each peak in the same multiple peak grouping, wherein the assignment of copy numbers to the identified peaks within each multiple peak grouping is adjusted until said plotting results in a substantially linear plot for at least one of the clones in the multi-clonal test sample.

In at least one embodiment, the expected ratios are calculated as the quantity k/2-1, where k is the assigned copy number, and the slope of the plot is assumed to be approximately one, e.g., between about 0.8 and 1.0, or between 0.85 and 0.90, based on assuming that the test sample (e.g., a cell line or germline sample) comprises a single clone. In these embodiments, the assignment of the normal peak (zero peak) of the centralization curve is adjusted to achieve this expected slope.

These and other features of the invention will become apparent to those persons skilled in the art upon reading the details of the methods, systems and computer readable media as more fully described below.

### BRIEF DESCRIPTION OF THE DRAWINGS

FIG. 1 is a schematic representation of an array.

FIG. 2 is an enlarged view of a portion of the array schematically shown in FIG. 1.

FIG. 3, an illustration of a plot of log ratio values versus chromosomal location represented by the probes from which the probe signals are read to calculate the log ratio values.

FIG. 4 illustrates events that may be carried out in one approach to eliminate or greatly reduce erroneous types of aberration calls.

FIG. 5 illustrates a histogram.

FIG. 6 illustrates fitting of Gaussian curves to peaks.

FIG. 7 illustrates a method of incrementally moving an axis representing a zero log ratio value, in the process of creating a centralization curve.

FIG. 8 illustrates a centralization curve created by the process described with regard to FIG. 7.

FIG. 9 illustrates a centralization curve.

FIG. 10 shows a series of events that may be carried out to identify copy numbers represented by different peaks in a centralization curve.

FIG. 11 illustrates the linear relationship between copy number and ratio values of a test sample to a reference sample.

FIG. 12 shows centralization curve plots of a distribution of log ratio values of probe signals from a primary tumor sample relative to a reference sample and a distribution of log ratio values of probe signals from a metastasis (e.g., secondary tumor) sample relative to the same reference sample.

FIG. 13 shows plots of the observed ratios of the peaks in FIG. 12 (Y-axis) versus the expected ratios according to the assigned copy numbers.

FIGS. 14A-14B illustrate an example of results from the use of a method described herein.

FIGS. 15A-15B illustrate an example of results from the use of a method described herein on the same data as that used with regard to FIGS. 14A-14B, but with centralization to a different peak.

FIG. 16 is a schematic illustration of a typical computer system that may be used in performing methods described herein.

### DETAILED DESCRIPTION OF THE INVENTION

Before the present systems, methods and computer readable media are described, it is to be understood that this invention is not limited to particular arrays, datasets, software or hardware described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a probe” includes a plurality of such probes and reference to “the array” includes reference to one or more arrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

A “test sample” as applied to CGH analysis, refers to a sample that is being analyzed to evaluate DNA copy number, for example, to look for the presence of genetic anomalies, or species differences, for example. A “reference sample” as applied to CGH analysis, is a sample (e.g., a cell or tissue sample) of the same type as the test sample, but whose quantity or degree of representation is known or sequence identity is known. As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample”, i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked into the sample.

A test sample and reference sample may both be contacted to a single array for co-hybridization therewith, wherein log ratios of signals from the two samples can be generated by reading the signals for the test sample on a first channel and reading signals for the reference sample on a two-channel analyzer. Alternatively, the test sample may be hybridized to a first array and the reference sample may be hybridized to a second array that is the same as the first array, and signals from each array may be read, and then compared as log ratios.

An “outlier region” refers to a region of values that is above or below a predefined threshold. Thus, for a predefined upper threshold value, an outlier region lies above the upper threshold value. For a predefined lower threshold value an outlier region lies below the lower threshold value.

An “outlier” is a value that is above or below a predefined threshold, depending upon whether the threshold is an upper threshold value or lower threshold value, respectively.

A “calibration array” refers to an array prepared with a normal male-female sample with no genetic abnormalities and hence no aberrations along the genome.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome X's (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

By “genomic source” is meant the initial nucleic acids that are used as the original nucleic acid source from which the probe nucleic acids are produced, e.g., as a template in the nucleic acid amplification and/or labeling protocols.

If a surface-bound polynucleotide or probe “corresponds to” a chromosomal region, the polynucleotide usually contains a sequence of nucleic acids that is unique to that chromosomal region. Accordingly, a surface-bound polynucleotide that corresponds to a particular chromosomal region usually specifically hybridizes to a labeled nucleic acid made from that chromosomal region, relative to labeled nucleic acids made from other chromosomal regions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays.

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

If a surface-bound polynucleotide or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

“CGH” or “Comparative Genomic Hybridization” refers generally to techniques for identification of chromosomal alterations (such as in cancer cells, for example). Using CGH, ratios between tumor or test sample and normal or control sample enable the detection of chromosomal amplifications and deletions of regions that may include oncogenes and tumor suppressive genes, for example.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein. In certain aspects, a CGH array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kbp, at least about 5 kbp, at least about 10 kbp, at least about 25 kbp, at least about 50 kbp, at least about 100 kbp, at least about 250 kbp, at least about 500 kbp and at least about 1 Mbp. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

In certain aspects, in constructing the arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences. In certain embodiments, one can have all of the probes directed to coding sequences. In certain other aspects, individual probes comprise sequences that do not normally occur together, e.g., to detect gene rearrangements, for example.

In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic (e.g., non-coding) regions of a nucleotide sample of interest. In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 bp or even 100,000 bp of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning.

Themed arrays also can be fabricated for use in gene expression assays, for example, to detect expression of genes involved in selected pathways of interest, or genes associated with particular diseases of interest.

In one embodiment, a plurality of probes on the array are selected to have a duplex Tₘ within a predetermined range. For example, in one aspect, at least about 50% of the probes have a duplex Tₘ within a temperature range of about 75° C. to about 85° C. In one embodiment, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In certain aspects, longer polynucleotides may be used as probes. In addition to the oligonucleotide probes described above, cDNAs, or inserts from phage BACs (bacterial artificial chromosomes) or plasmid clones, can be arrayed. Probes may therefore also range from about 201-5000 bases in length, from about 5001-50,000 bases in length, or from about 50,001-200,000 bases in length, depending on the platform used. If other polynucleotide features are present on a subject array, they may be interspersed with, or in a separately-hybridizable part of the array from the subject oligonucleotides.

In still other aspects, probes on the array comprise at least coding sequences.

In one aspect, probes represent sequences from an organism such as Drosophila melanogaster, Caenorhabditis elegans, yeast, zebrafish, a mouse, a rat, a domestic animal, a companion animal, a primate, a human, etc. In certain aspects, probes representing sequences from different organisms are provided on a single substrate, e.g., on a plurality of different arrays.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, the control target molecules in a population are present at a level comparable to a haploid amount of a gene represented in the target population. In another aspect, the control target molecules are present at a level comparable to a diploid amount of a gene. In still another aspect, the control target molecules are present at a level that is different from a haploid or diploid amount of a gene represented in the target population. The relative proportions of complexes formed labeled with the first label vs. the second label can be used to evaluate relative copy numbers of targets found in the two samples.

In certain aspects, test and reference populations of nucleic acids may be applied separately to separate but identical arrays (e.g., having identical probe molecules) and the signals from each array can be compared to determine relative copy numbers of the nucleic acids in the test and reference populations.

Methods to fabricate arrays are described in detail in U.S. Pat. Nos. 6,242,266; 6,232,072; 6,180,351; 6,171,797 and 6,323,043. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685; 6,221,583 and elsewhere).

An array is “addressable” when it has multiple regions of different moieties, i.e., features (e.g., each made up of different oligonucleotide sequences) such that a region (i.e., a “feature” or “spot” of the array) at a particular predetermined location (i.e., an “address”) on the array will detect a particular solution phase nucleic acid sequence. Array features are typically, but need not be, separated by intervening spaces.

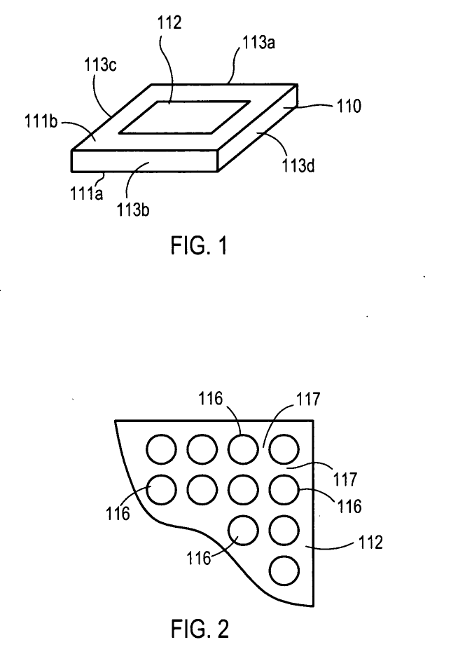

An exemplary array is shown in FIGS. 1-2, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a surface of the substrate and depending on the use of the array, any or all of the arrays may be the same or different from one another and each may contain multiple spots or features. The one or more arrays 112 usually cover only a portion of the surface 111b, with regions of the surface 111b adjacent the opposed sides 113c, 113d and leading end 113a and trailing end 113b of slide 110, not being covered by any array 112. A surface 111a of the slide 110 does not carry any arrays 112. Each array 112 can be designed for testing against any type of sample, whether a test sample, reference sample, a combination of them, or a known mixture of biopolymers such as polynucleotides. Substrate 110 may be of any shape, as mentioned above.

As mentioned above, array 112 contains multiple spots or features 116 of oligomers, e.g., in the form of polynucleotides, and specifically oligonucleotides. As mentioned above, all of the features 116 may be different, or some or all could be the same. The interfeature areas 117 could be of various sizes and configurations. Each feature carries a predetermined oligomer such as a predetermined polynucleotide (which includes the possibility of mixtures of polynucleotides). It will be understood that there may be a linker molecule (not shown) of any known types between the surface 111b and the first nucleotide.

Substrate 110 may carry on surface 111a or elsewhere, an identification code, e.g., in the form of bar code (not shown) or the like printed on a substrate in the form of a paper or plastic label attached by adhesive or any convenient means. The identification code contains information relating to array 112, where such information may include, but is not limited to, an identification of array 112, i.e., layout information relating to the array(s), etc.

In the case of an array in the context of the present application, the “target” may be referenced as a moiety in a mobile phase (typically fluid), to be detected by “probes” which are bound to the substrate at the various regions.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

“Hybridizing” and “binding”, with respect to nucleic acids, are used interchangeably.

A “design file” is typically provided by an array manufacturer and is a file that embodies all the information that the array designer from the array manufacturer considered to be pertinent to array interpretation. For example, Agilent Technologies supplies its array users with a design file written in the XML language that describes the geometry as well as the biological content of a particular array.

“design pattern” is a description of relative placement of features, with annotation. A grid template or design pattern can be generated from parsing a design file and can be saved/stored on a computer storage device. A grid template has basic grid information from the design file that it was generated from, which information may include, for example, the number of rows in the array from which the grid template was generated, the number of columns in the array from which the grid template was generated, column spacings, subgrid row and column numbers, if applicable, spacings between subgrids, number of arrays/hybridizations on a slide, etc. An alternative way of creating a grid template is by using an interactive grid mode provided by the system, which also provides the ability to add further information, for example, such as subgrid relative spacings, rotation and skew information, etc.

A “property” of an array, as used herein refers to a characteristic of an array that may be measured through analysis and calculation based on signals received during reading (e.g., scanning or other method of obtaining signals from) the array, and which may be used as a measure of quality of the array. Properties include, but are not limited to, noise, signal-to noise, background signal, signal intensity, uniformity/non-uniformity, population outlier, saturated feature, etc.

A “probe signal”, “probe value” or “probe signal value” refers to the observed signal obtained from the probe, i.e., the signal from a probe bound to a target, including “true” signal (i.e., from the target that the probe was designed to bind with) and offset, such as from cross-hybridization and other noise factors, including background.

When one item is indicated as being “remote” from another, this is referenced that the two items are not at the same physical location, e.g., the items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

### Methods, Systems and Computer Readable Media

The present invention provides methods, systems and computer readable media for processing CGH data in ways that reduce ambiguities in results compared to results provided by currently existing methods and systems. In one aspect of the present invention, methods, systems and computer readable media are provided for estimating the minimum difference in log ratio of test sample signals to reference sample signals from a “normal” log ratio value that should be considered biologically significant to identify an aberration.

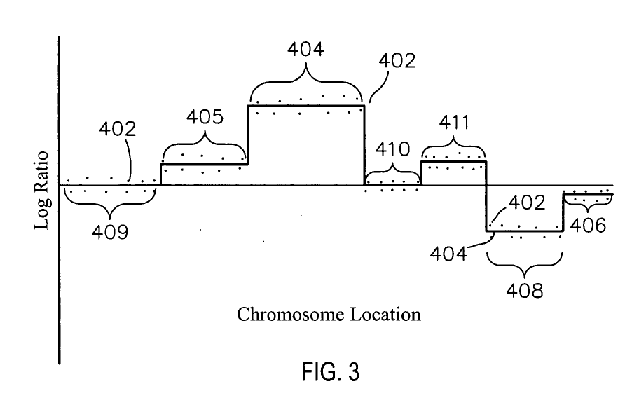

Referring to FIG. 3, an illustration of a plot 400 of log ratio values between two channels from a CGH array (or a plot of log ratio values comparing signals from two arrays having identical probes, as described above, where a test sample is hybridized to one of the arrays and a reference sample is hybridized to the other array, for example) is provided. The probe signals from the array have been rearranged to correlate to their positions represented on the chromosomes in the plot 400, thereby mapping them to the chromosome locations (chromosomal coordinates represented by each probe, respectively), with log ratio values of the two channels/two arrays for each location/probe represented by data points 402. That is, the probes, as arranged, are capable of hybridizing, under stringent conditions, to consecutive positions along a chromosome. Note that consecutive does not necessarily mean directly adjacent to, as consecutive arrangement is defined along a consistent direction, e.g., such as from one chromosome arm to another along the same chromosome. An average log ratio value line 404 has been drawn based upon the plotted points 402, with discontinuities connected by vertical lines. FIG. 3 shows data points 402 after centralization as described in co-pending, commonly owned application Ser. No. 11/338,515.

As noted above, even after centralization of the CGH data, some chromosomal regions, or even entire chromosomes end up with average reported log ratios slightly different from zero. Various different factors have been postulated to be the cause of this phenomenon, some of which were described above, and others being thought to originate from the biology of the samples themselves. Regardless of the cause(s), it would be advantageous to be able to identify those regions which are not truly reporting aberrations in copy number, but which are nevertheless reporting non-zero average log ratio values.

Regions 405 and 406 in FIG. 3 are exemplary of this phenomenon. Where the genetic material is “normal” and no amplification or deletion has been reported, the average log ratio signal is expected to be zero, since the fold number should be the same in both channels/arrays. When one channel/array represents abnormal tissue, such as cancer tissue, for example, and the other channel/array is a control channel representing normal or non-cancerous tissue, then the regions in which amplification or deletion has occurred in the cancerous or otherwise abnormal tissue shows up by log ratio values that deviate from zero, e.g., a value around +1 for an amplification of two, such as the amplification region 407 shown in FIG. 3 or a significantly negative value indicating a deletion, such as illustrated in region 408 in FIG. 3. The amount of the negative value plotted depends on the average ploidy of the sample, the clonality (i.e., number of clones and the fraction of cells that each clone represents, respectively) of the sample, and the copy number of the deletion or amplification. For example, if the average ploidy is two, and the sample is monoclonal, a 1:2 deletion will show a log ratio of about −0.7 to about −1.0. As another example, where the average ploidy is two, and the sample is monoclonal, a 3:2 amplification (i.e., where the copy number in the abnormal tissue sample is three and the copy number of the normal tissue sample is 2) will show a log ratio of about 0.4 to about 0.6.

However, for regions such as regions 405 and 406, even though they are only slightly different from zero and thus likely not reporting true aberrations, such regions may appear to be statistically significant, judged by the average probe-to-probe variation observed for the sample ratios, even though their difference from zero does not indicate any copy number variation in the test sample relative to the reference sample in this region. The reason for this is that aberration calling algorithms, such as ADM-1, for example (see Lipson et al., “Efficient Calculation of Interval Scores for DNA Copy Number Data Analysis”, Journal of Computational Biology, March 2006, Vol. 13, No. 2 : 215-228, which is incorporated herein, in its entirety, by reference thereto), scores regions (or intervals) as a function of the average log ratio of the region (also referred to as “interval”) as well as the square root of the length of the region (interval). For example, the score in the ADM-1 routine is calculated as:

$$S ( I ) = \frac{{\sum }_{j ε I}^{ } {v}_{j}}{\sqrt{\vert I \vert}} \qquad ( 1 )$$

where:

### S=score;

### I=interval;

### S(I)=the score for the interval; and

vⱼ=the average log ratio signal value generated from the probes targeting subsequence j in interval I.

Thus, it can be seen that the score calculated in the above approach is proportional to the average log ratio and also to the square-root of the length of the interval. Consequently, when a region such as region 405 or 406 that is reported as only slightly different from zero is a relatively long region, it will be scored as a relatively high score even though the average log ratio is very small. This results in potential errors in calling aberrations for regions that are long regions with low average log ratio values (i.e., close to, but not equal to zero, either negative or positive) but with no biological significance, i.e. “false positives”.

Each data point on the plot in FIG. 3 can be called to be either normal or aberrant. If a data point is called normal, this means that it has been determined that the genomic position corresponding to the data point exhibits a copy number in the test sample that is equal to the copy number in the reference sample. Conversely if a data point is called aberrant, this means that the log ratio of the probe signal of the test sample to the probe signal of the reference sample indicates that the copy number of the test sample is not equal to the copy number of the reference sample at the genomic location corresponding to the probe. If a histogram of log ratio values of all probes over the genome is generated, then any log ratio values within the width of the peak that is considered to represent the “normal” data values, i.e., the “zero” peak, should be treated as non-aberrant. Accordingly, a tolerance value C can be calculated, such that the data points with reported log ratios within ±C of the zero value assigned to the data will be called normal, as they truly do not represent a biologically significant difference in copy number from normal. Thus, values of +C and −C can serve as upper and lower thresholds, within which log ratio values will be called normal. Put another way, if the absolute value of a log ratio value is less than or equal to C, then that log ratio value is considered to be a normal, non-aberrant call.

In order to eliminate or greatly reduce the incidence of erroneous aberration calling, one approach taken in the present invention analyzes the probe signals of those probes determined to belong to statistically significant aberrant intervals as a function of the log ratio value chosen as the “zero” log ratio value. This “zero” value selection may be made by any of the techniques described previously herein. Preferably, however the “zero” value will have been determined by including the centralization step described in application Ser. No. 11/338,515, with the understanding that the choice of which peak of the centralization curve is selected as the appropriate “zero” peak should be made according to the methods described herein, when they differ from the methods previously described.

When analyzing CGH data to call aberrations, regions of the chromosomes exhibiting substantially constant log ratios can typically be identified. For example, in the data plotted in FIG. 3, regions 405-411 would be identified as regions exhibiting substantially constant log ratios. Regions 409 and 410 clearly show a zero log ratio value and would be called “normal” upon analyzing these regions. Regions 407 and 408 may be biologically significant in that they represent aberrant regions and would therefore accurately be called out as aberrations. However, as noted above, regions 405, 411 and 406 may not be biologically significant, since they deviate from zero by only a small amount, yet they might still be erroneously called as aberrations by the scoring algorithms as described in application Ser. No. 11/338,515.

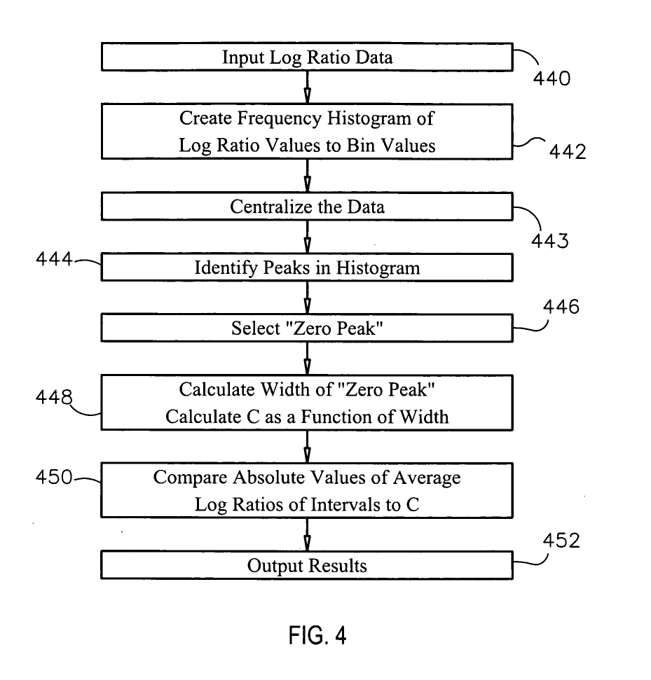

FIG. 4 illustrates events that may be carried out in one approach to eliminate or greatly reduce the erroneous type of aberration calling referred to above. At event 440, log ratio data calculated from a test sample and a reference sample hybridized to one or more arrays is inputted. As already noted, these data points will have already been normalized using one or more of the techniques referred to above.

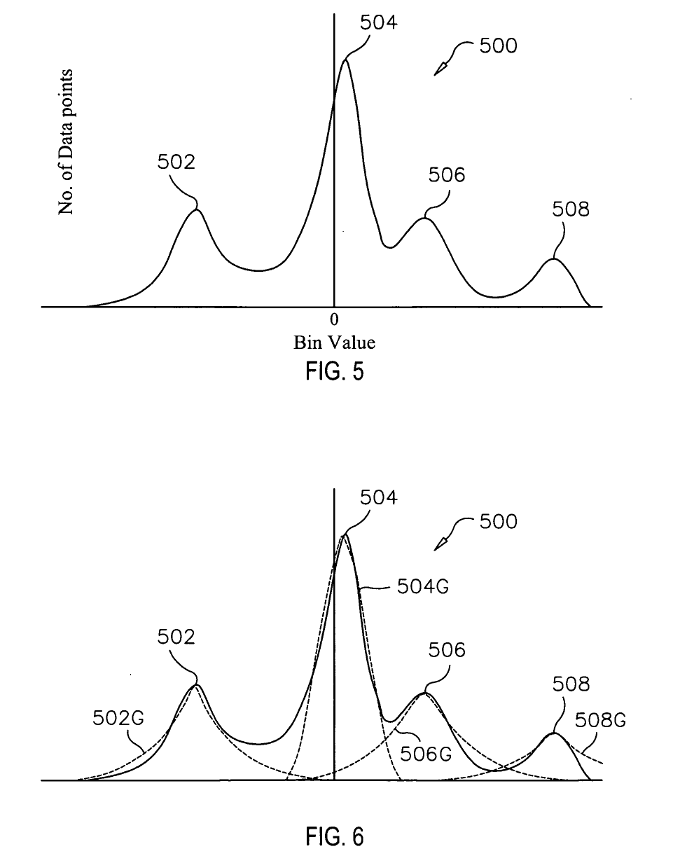

At event 442 a histogram is created that plots the number of occurrences (where each occurrence is representative of a log ratio value from a pair of probe signals, i.e. a data point) of log ratio values in each bin, wherein a bin spans a predetermined range of log ratio values. For example, by selecting a bin size of 0.02, log ratio values would be binned in log value ranges of 0.00 to 0.02, 0.02 to 0.04, 0.04 to 0.06, . . . etc. and 0.00 to −0.02, −0.02 to −0.04, −0.04 to −0.06, . . . etc. This binning is analogous to smoothing the data with a window size of the bin size selected. An example of such a histogram 500 is shown in FIG. 5. Although the curve is shown as continuous, it is noted that discrete data points for numbers of data points versus bin values are plotted and these plotted points can then be used to plot the approximately continuous curve shown. The peak representing the “normal” log ratio values (i.e., those representing no aberrations, where log ratio is expected to be zero) may not be centered exactly on zero, for reasons already discussed, even though dye normalization and other normalization processing may have already been performed on the data. In this case, the data may optionally be further processed for centralization as described in application Ser. No. 11/338,515 at event 443 to attempt to align the most prominent peak (e.g., peak 504 in FIG. 5) with a zero log ratio value. Event 443 can be described as a linear renormalization step specific to CGH data. To perform this secondary normalization between the channels (e.g., red and green channels from a single array, or single channels from two different arrays), which is also referred to as centralization, a curve is generated and referred to as a centralization curve. For example, the centralization curve 500 of FIG. 5 would indicate a new value for “zero” along the x-axis where it aligns with peak 504. After this centralization, the entire plot is shifted to the left to align the highest peak at the zero value, according to the method described in application Ser. No. 11/338,515. That method assumes that most of the genome is non-aberrant, as this is most always the case. Accordingly, the highest peak in the histogram (i.e., peak 504 in FIG. 5) was assumed by the method described in Application Serial No. 11/338,515 to be the peak representing those data points representing no aberration, and this peak was assigned the value of zero (the “zero peak”). The present invention describes methods by which, in some cases, this assumption can be obviated.

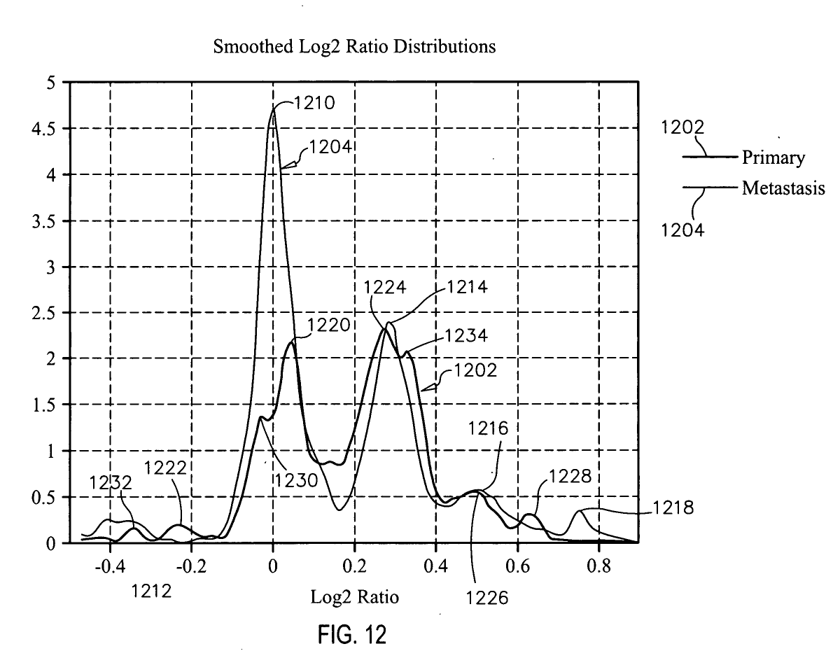

Even after centralization, the potential for erroneous aberration calling for regions that have a long length and a small deviation from zero exists, for reasons already noted. Centralization is a linear normalization that shifts all of the log ratios in one direction (either positive or negative) to align the log ratio of the assigned “non-aberrant” peak as the best fit for the zero value. In this approach to addressing this potential problem, at event 444, the peaks in the histogram 500 are identified using any of many peak finding routines, such as one provided in Matlab, for example, among many others. More specifically, the most prominent N peaks are identified, wherein N is an integer. Typically, “N” is determined by the number of peaks found by the peak finding algorithm, with a user settable limit often being included to prevent an unworkable number of peaks being provided in the result for pathological cases. For example, a user may set an upper limit for “N” as 20, or 18, or some other integer thought to limit an unwanted number of peaks that would typically not all correspond to copy numbers. In one approach, the most prominent peak can be identified (i.e., greatest magnitude along the Y-axis), along with neighboring peaks that are separated from this most prominent peak by sufficient distances such that they could possibly represent increments of copy numbers. In the exemplary histogram 500 of FIG. 5, N=4, as four prominent peaks are identified. Peak 504 is identified as the most prominent peak and four peaks in all are identified (502, 504, 506, and 508) during event 444. In the centralization curve or FIG. 12, six peaks are identified.

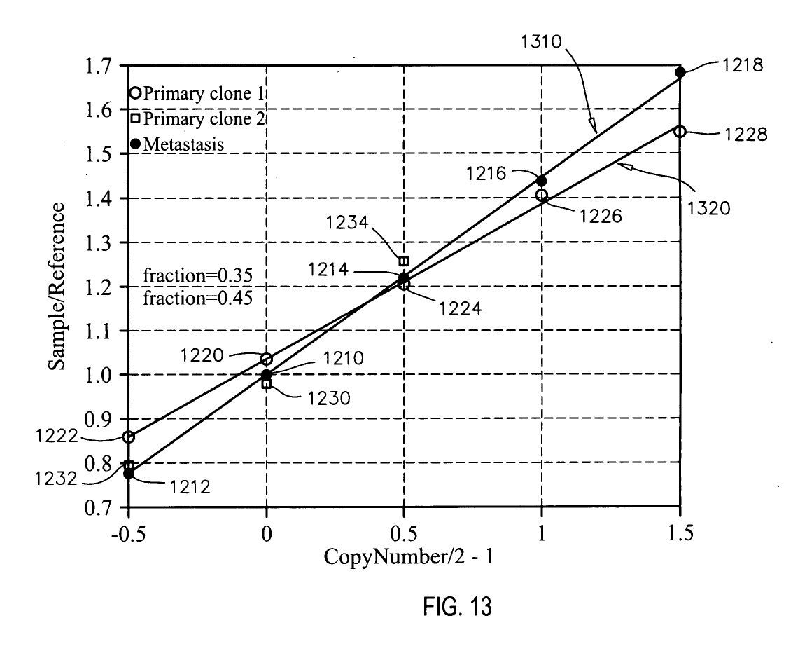

The most prominent peak is typically the peak formed by the log ratio values considered to be “normal” (i.e., non-aberrant, wherein the test sample has a copy number equal to the reference sample, which has the normal, expected number of copies), since, most often, a global maximum is attained at the most common ploidy of the genome, and this is most often the same as the normal ploidy. However, this is not always the case, and in this small percentage of times when the most prominent peak is not representative of normal, the “normal” peak can be selected heuristically. One heuristic approach is to select the peak that is most to the left in the centralization plot (i.e., lower copy number), when two or more peaks appear that are both prominent relative to other peaks. If there is remaining uncertainty about which peak corresponds to normal copy number, the best fit to the slope (described in regard to FIGS. 11 and 13 below) can be used to choose among them, if the sample is known to be monoclonal. If uncertainty remains about the predominant ploidy of the test sample, confirmation can be made by using other techniques such as spectral karyotyping (SKY) or fluorescence in situ hybridization (FISH). If the predominant ploidy of the test sample is known, based on non aCGH results, the user may select a peak from the centralization plot to correspond to the normal copy number. By at least one of these methods, a peak is selected at event 446 as being representative of the “zero peak”.

Next, the width of the peak identified in event 446 is calculated. Since the peak found/identified at event 446 represents data that exhibits no aberration (i.e., is normal), then all log ratio values within the peak should be considered normal, and the tolerance, C, should be defined as being at least as big as the width of the peak, so as to avoid identifying normal data as aberrant. Various mathematical measurements may be employed to represent the width of a peak, for example, the width of a peak may be represented by the variance, standard deviation, or range of the peak. The width may also be represented by any measure standing in known relation to any of those aforementioned quantities. Therefore, in the context of the example of FIG. 5, the width of the peak 504 is calculated at event 448, that is the variance, standard deviation, range, or other measurement of the tendency of the peak 504 to spread is calculated. Thereafter, the tolerance, C is calculated, wherein the value of C is a function of the width, e.g., C=width, or C=k*width, where k is a constant, etc.

At event 450, average log ratios of all intervals obtained from the aberration detection algorithm are compared with the calculated tolerance threshold value C. If the absolute value of the mean log ratio of any interval is less than or equal to C, then that interval is considered non-aberrant and is called normal and/or non-aberrant. Otherwise, the region is considered aberrant and is called aberrant. Accordingly, the calculated tolerance value can be used to eliminate regions that would otherwise have been called aberrant, but which do not have biological significance (e.g., are false positives, that are non-zero due to the drift or deviation from zero described above).

At event 452, results are outputted for viewing and use by a user. For example, outputted results may display the tolerance value C. Further, the log ratio data may be analyzed by an aberration calling algorithm, such as ADM-1 or the like, using C as a threshold (+C=upper threshold, −C=lower threshold) so that any genomic region that would otherwise have been called aberrant by the scoring method of the aberration calling algorithm, if having an average log ratio value ≦C and ≧−C, is called normal or non-aberrant. Additionally, data points/regions that are called normal may be outputted as results and labeled as “normal”. Still further, regions of aberrant data points can be identified and called aberrant, and/or plots of the same may be outputted. Also, identification of those regions called normal, but not having a zero average log ratio value, yet falling within the tolerance range, can be identified as such. Still further, copy number values of aberrant data points can be calculated and outputted.

In peak finding as described above, it can be assumed that the central portion of the data plotted in the histogram 500 is mainly composed of N Gaussian curves with centers close to the detected peak centers, each curve having equal variance. FIG. 6 illustrates a process of fitting N Gaussian curves to N identified peaks, wherein the histogram is the same as that shown in FIG. 5 and N=4. Each of the Gaussian curves is defined by an amplitude, a mean, and a standard deviation or variance. Thus FIG. 6 illustrates the central portion of the curve 500 being fitted to the summation of the four Gaussian distributions 502G, 504G, 506G and 508G (shown in dashed lines) fit to respective ones of the peaks 502, 504, 506, and 508. According to some embodiments, the process of fitting the Gaussian curves 502G, 504G, 506G, and 508G to the histogram curve 500 is constrained to assume that the standard deviation/variance of each of the curves 502G, 504G, 506G, and 508G is the same. According to some embodiments, some of the parameters (e.g., amplitude) can be determined by minimization of the error function, while other parameters, such as peak position and variance, for example, can be determined using the Nelder-Mead simplex (direct search) method of fitting, to optimize these parameters.

Once the fit has been completed, the standard deviation, variance, or some value standing in known relation thereto can be used to calculate C as described with regard to event 448 above. In one embodiment, the software embodying the process described above may prompt a user to enter a desired confidence level (e.g., via a user interface) of the genomic sites identified as aberrant, and may then assign C based upon the desired confidence level and the variance/standard deviation. For example, assuming that the user entered a desired confidence level of 95%, then C may be assigned a value of three times the standard deviation of the Gaussian curve fit to the peak identified as corresponding to “zero” or “normal”. The comparisons and outputs can be performed, as in events 450 and 452, based upon this value of C.

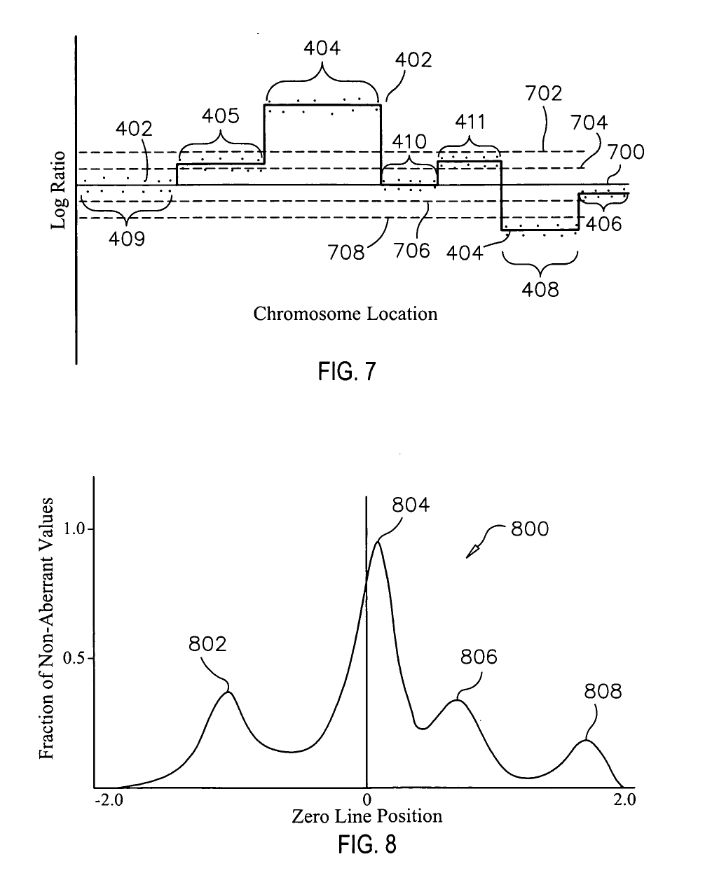

Another approach to creating a centralization curve that avoids the necessity of selecting a bin size to generate a histogram is now described. FIG. 7 shows the same plot of log ratio values plotted with regard to chromosome location in a genome as described above with regard to FIG. 3 but is referred to for demonstrating the current approach to creating a centralization curve. Using this approach, the log ratio data values are analyzed using the original zero value of the plot (which may be a first estimate of a zero value after initial normalization of probe signal values, indicated by line 700 in FIG. 7) to make aberration calls (such as by using ADM-1, or other aberration calling algorithm, for example. The fraction of non-aberrant (normal) log-ratio values (data points for log ratios of probe signals as described previously) is then calculated for all data points in the plot. This fraction is paired with the “zero” value, in this case 0.0. The zero line is then shifted (either upward or downward, as a series of these shifts in both directions will be performed, as described hereafter) by a predetermined increment, which may is greater than 0.0 and less than 0.01 or between about 0.01 and 0.10, or between about 0.1 and 0.2, or some other increment that may be chosen and which may depend in part upon the particular characteristics of the data set being analyzed, but need not be.

In any event, the log ratio data values are again analyzed using the next zero value line of the plot of the plot (which is line 702 in the first iteration in FIG. 7). Again the fraction of non-aberrant log ratio values is determined and paired with the current position of the zero value line. This process is repeated for each increment up to a predetermined end value for stopping the incrementing of the zero line in one direction (e.g., 2.00, or some other predefined value) and then the process is repeated in the opposite direction, each time incrementing the zero line by the same predefined increment and calculating the resulting fraction of non-aberrant values. The end point in the opposite direction may be the same absolute value as the stopping value in the first direction (e.g., in this example, −2.00) or may be some other predetermined value (e.g., −1.50). In any event, upon completion of the iterations, a set of paired values is returned, each pair including the position of the zero line on the Y-axis in FIG. 7 and a fraction value of the number of non-aberrant values determined for that position. These paired values are then plotted to form a centralization curve, as illustrated in FIG. 8. Further details about creating a centralization curve for purposes of centralizing the data can be found in application Ser. No. 11/338,515.

FIG. 8 illustrates a centralization curve 800 that is created by the process steps described above with regard to FIG. 7, but not necessarily from the data values shown in FIG. 7. Rather, the curve in FIG. 8 is created from the same data values used to create the histogram of FIG. 5. However, FIG. 8 plots the fraction of non-aberrant values found versus the zero line position, where the zero value shown in FIG. 8 is the original value line as plotted. The zero line can then be shifted to coincide with the peak of the most prominent peak (804 in FIG. 8), or to correspond to another peak consider to be the most likely “normal” peaks, using any of the techniques described above. Note that the shape of curve 800 is very similar to that of curve 500, as is expected. There can be some variation between these two techniques, and one factor for such differences can be the size of the bins used to generate the histogram in FIG. 5. Since the same aberration-calling algorithm can be used to generate a centralization curve in this manner as is used in making the final aberration calls after a zero value has been located and a peak has been processed to determine a tolerance value, then any idiosyncrasies in the aberration calling algorithm will be the same when creating the centralization curve as when calling aberrations. This reduces the degrees of freedom in processing by one, as compared to the technique described above where a histogram is generated. Accordingly, it is believed that this technique will be even more robust in minimizing the number of erroneously called aberrations.

After creation of the centralization curve according to this approach, events 444 to 452 can be carried out to more accurately call aberrations.

In addition to identifying the zero peak (i.e., the peak corresponding to normal values copy numbers, two in many instances) it would also be desirable to identify other peaks that correspond to other copy numbers. In this way, not only can the predominant ploidy of the sample be identified, but the copy numbers of aberrant regions of the genome can also be identified. For a single clone, adjacent peaks will correspond to integral numbers of copies being reported, but this is not the case when a sample contains multiple clones. Different clones may have different copy numbers at the same genomic location, so that the peak resulting from averaging the probe signal ratios, for all clones relative to the reference sample, may be a non-integer value.

In order for a cancer to metastasize, a multiplicity of certain conditions need to be met. Cancer begins when the genome begins to function abnormally and aberrations in the genetic material occur. Many different mistakes can be made in the genetic material of a cell before it becomes a cancer cell. To become a cancer, cells must be growing out of control. These cells must be viable and must be able to divide, at a supernormal rate. Before the cancer can metastasize it must be able to invade into the blood stream so as to be transported to other locations in the body. It is usually a breakdown of the regulatory machinery of the cell that allows these events to occur, but they do not all occur at once. Accordingly, in the process of the cellular machinery breaking down, many mistakes in genomic copying are made that are byproducts of the regulatory machinery breaking down, and which are irrelevant to the cancer.

Consequently, it is important for cancer researchers to discern which genomic aberrations are necessary for the cell to become a cancer cell, versus those which are merely incidental. Also, there may be precancerous cells present in a tumor, which are not fully able to metastasize. Thus, in the process of tumor formation, there are often different cell populations (different clones) residing in that tumor. For example, a typical tumor may have between one and five or six different clones. These different cell populations (clones) that are viable are all continuing to divide and compete with each other for resources (nutrients, etc). Eventually, one clone begins to overtake the other clones, through the process of natural selection, and begins to dominate the tumor. With further development, this dominant clone may develop the ability to metastasize, where cells can break free of the tumor, get into the blood stream and travel to other organs to propagate there.

Thus, when analyzing primary and secondary tumors, depending upon where the sample is taken from the primary tumor, different results can occur, as the makeup of the tumor may vary by location with respect to different clones. In addition, tumor samples often include many cells from normal tissue adjacent to the cancerous tissue. However, when analyzing a metastasized (secondary) tumor, only one clone will typically be found, because it is common for a single clone to evolve the genomic properties necessary for metastasis. Samples from a metastatic tumor, though, can also include normal cells. Therefore samples from a metastatic tumor are highly likely to consist of a mixture of cells of a single aberrant (i.e., cancer) genotype, admixed with normal cells, whereas samples from the primary tumor site may consist of several aberrant clones, also admixed with normal cells.

By creating a centralization curve by any of the techniques described above, peaks can be plotted that are representative of copy number changes in the cancerous tissue sample relative to a reference sample. It is assumed that each of the identified peaks (peaks identified such as described with regard to event 444, regardless of how the centralization curve is formed) represents a particular copy number of one or more regions of the sample. For example, for diploid normal samples, the zero peak represents a copy number of two, the peak to the left of the zero peak is expected to represent a copy number of one, the peak to the right of the zero peak is expected to represent a copy number of three, etc.

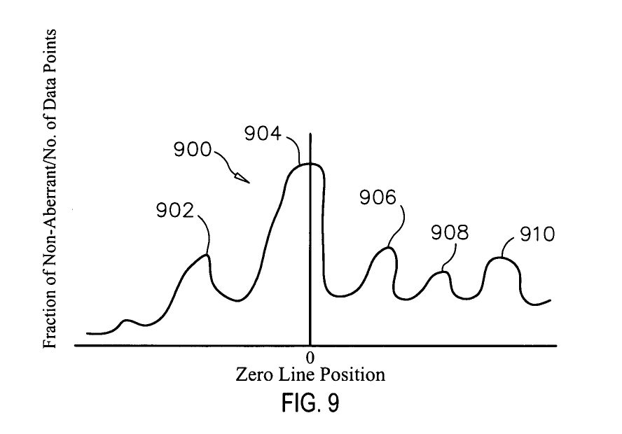

FIG. 9 illustrates a centralization curve 900. Whether created by the histogram method described above, or the method of incrementally moving the zero line and tallying non-aberrant data values, or some other method, when analyzing a tissue sample, it is generally assumed that the cells that are exhibiting aberrations, if any, are also diluted with normal cells that do not exhibit these aberrations. Therefore the peaks displayed in the resulting centralization curve may not be spaced at regular intervals that would indicate copy numbers of 1, 2, 3, . . . etc., which would be the case if the tissue sample were purely composed of a single cell line exhibiting aberrations. In order to call the copy number for each peak (and thus for ranges of log ratio values that will be read by an aberration-calling algorithm) an assignment of copy numbers to the peaks must is first be made.

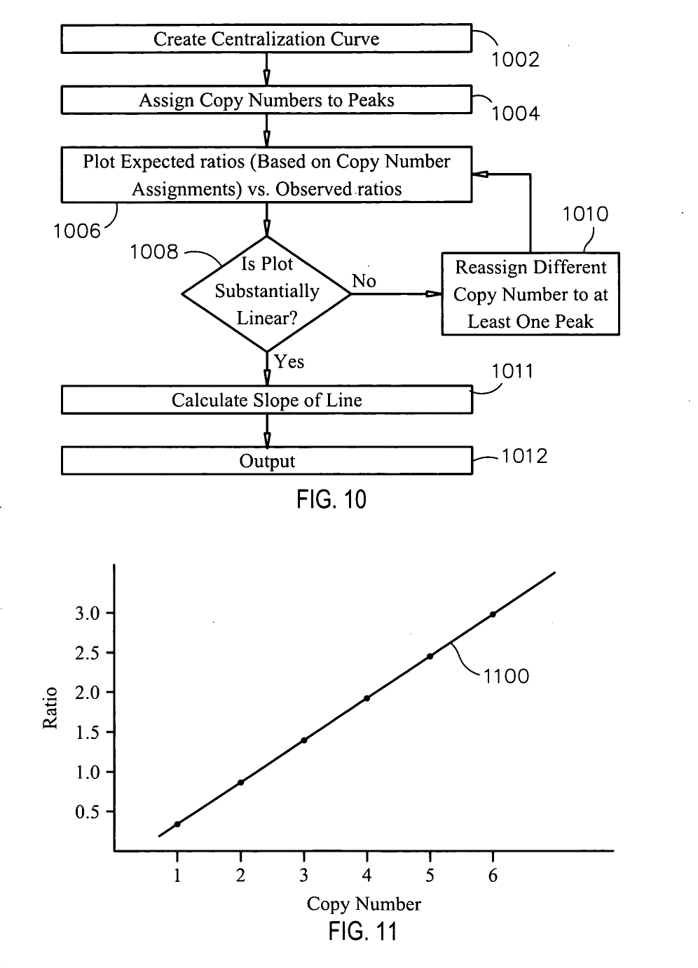

FIG. 10 shows a series of events that may be carried out to identify copy numbers represented by different peaks in a centralization curve. After creation of the centralization curve at event 1002, and peak finding in a manner as described above, an initial assignment of copy numbers to peaks in the centralization curve is made at event 1004. This initial assignment may include assigning a copy number of two (or the copy number of the “normal” reference sample) to a “zero peak” in any of the ways described above, and then assigning copy numbers incrementally, with integers to the left and right (i.e., adjacent peak to the left of zero peak has copy number of one when zero peak has copy number of two, adjacent peak to right of zero peak has copy number of three when zero peak has copy number of two, adjacent peak to the right of peak assigned copy number three is assigned copy number four, . . . etc.) Alternatively, the left-most peak may be assigned a copy number of one and then each peak to the right may be assigned a copy number that is incrementally one greater than the copy number of the peak immediately to the left of it. For example, in FIG. 12, the “zero peak” 1210 has been selected for the metastatic centralization curve 1204 and assigned a copy number of two, so peak 1212 has been assigned a copy number of one, peak 1214 has been assigned a copy number of three, peak 1216 has been assigned a copy number of four, and peak 1218 has been assigned a copy number of five. Likewise, in FIG. 9, the “zero peak” 904 has been selected and assigned a copy number of two, so peak 902 has been assigned a copy number of one, peak 906 has been assigned a copy number of three, peak 908 has been assigned a copy number of four, and peak 910 has been assigned a copy number of five.

If the identified peaks do in fact represent regions with sequential copy numbers, then the log ratio values at which the peaks 902, 904, 906, 908 and 910 (or 1212, 1210, 1214, 1216 and 1218, for example) appear will satisfy a linear relationship. If the test sample is monoclonal, then the plot will have a slope close to, but less than one, when the log ratio values are converted to ratio values and plotted against copy number. The expected value of the measured slope for samples with a nominal slope of 1.0 is characteristic of the aCGH assay method. For the aCGH assays illustrated herein, the measured slope is specified to be greater than 0.80, and typically between about 0.85 and about 0.95, for samples with a true slope of 1.0. FIG. 11 illustrates this linear relationship, wherein ratios plotted against copy numbers form the line 1100. Generally, the expected ratio of a mixture of aberrant cells (single clone) with normal cells is given by: ratio=(k/2-1)a+1, where “a” is the fraction of tumor cells and “k” is the expected copy number of the tumor cells. A derivation of this relationship follows:

$$y = \frac{ak + ( 1 - a ) 2}{2}$$

where
y is the observed ratio of test sample probe signals to reference sample probe signals;
2 in the denominator represents the number of copies in the reference sample (which is always 2 for diploid reference samples, but may differ for reference samples of non-mammalian species, such as plant cells, for example);
a is the fraction of the test sample that is aberrant;
1-a is the fraction of the test sample that is normal (non-aberrant) and which is therefore multiplied by 2 (assuming normal tissue is diploid);
and
k is the assigned copy number for the peak.
Reorganizing the equation:

$$2 y = a ( k - 2 ) + 2 ; and$$

$$y = a ( \frac{k}{2} - 1 ) + 1$$

Comparing to the equation for a line, i.e., y=ax+b, where a is the slope of the line and b is the intercept, this gives (k/2-1) as the expected ratio value on the x-axis of the plot, with a, the fraction of the sample consisting of the aberrant clone, as the slope of the plot, and +1 as the y-intercept.

If “k” is correctly assigned to each of the observed peaks at event 1004, then a plot of the ratios of the peaks versus (k/2-1) will be linear, with the slope of the line=a, the fraction of aberrant cells in the sample. Accordingly, after assigning copy number at event 1004, the ratios (log ratio values where peaks appear are converted back to ratio values) of the peaks are plotted against the expected ratios (a function of their assigned copy numbers, i.e., (k/2-1)). The resulting plot is then reviewed to determine whether it is substantially linear at event 1008. There are many ways of determining whether the plot is substantially linear. One method is subjective calling, after review by a human, but this is typically not the best method. Generally, the fit is concluded to be “substantially linear” if fits based on different assumptions (from the fit deemed to be substantially linear) yield higher residuals than those achieved with the fit being called substantially linear. However, there are many other methods as would be readily apparent to those of ordinary skill in the art. A typical basis for concluding that a fit is linear is that any non-linear fit, which perforce requires at least one more adjustable parameter than a linear fit, doesn't achieve greater statistical reliability than a linear fit. If the plot is concluded to be not substantially linear at event 1008, then at least one reassignment of copy number values to the peaks is made at event 1010 to try and improve the linearity of the resulting plot and plotting is again performed at event 1006. Iterations of events 1006, 1008 and 1010 can be performed until the linearity of the resulting plot has been optimized, or until a human reviewer is satisfied that the resultant plot is substantially linear. In any event, once event 1008 has been satisfied regarding the production of a substantially linear plot, then the slope of that plot can be calculated at event 1010 to determine the fraction of the aberrant cells relative to the total number of cells in the test sample.

The plot will be substantially linear if an only if the assigned copy numbers k used to generate the plot are sequential. However, confirmed substantial linearity doesn't show that the assigned copy numbers are correctly assigned, just that they are sequential. The linearity of the plot can be used to prove the consistency of the copy number assignments, since if the plot is not substantially linear, then the copy number assignments cannot be correct, and to affirm the presence of multiple aberrant clones, as demonstrated in FIG. 13, for example. However, the substantial linearity of the plot cannot be used to confirm the correctness of the assignment of centralization peaks to absolute copy numbers. Assignment or centralization peaks to absolute copy numbers necessarily requires additional assumptions, such as heuristics or results from other than aCGH data (e.g., SKY, FISH, etc.).

At event 1012, the fraction can be outputted numerically for viewing and use by a user, such as by displaying it on a user interface/display, printing it out on a printer, or saving it to a storage device, among other ways of outputting. Additionally, or alternatively, the log ratio values of the peaks and their assigned copy numbers can be outputted in any of the same manners. Also, the centralization plot showing the peaks, their log ratios and assigned copy numbers can be outputted, and/or the linear plot that was considered to be substantially linear.

FIG. 12 shows centralization curve plots of a distribution 1202 of log ratio values of probe signals from a primary tumor sample relative to a reference sample and a distribution 1204 of log ratio values of probe signals from a metastasis (i.e., secondary tumor) sample relative to the same reference sample. The distribution of log ratios reflects the number of copies of different regions of the genome. After centering to the (assumed) diploid peak, the next (presumably triploid) peak appears at log₂ ratio=0.28, instead of the 0.59 expected for a 3/2 ratio (i.e., log₂(1.5)=0.59). From this it can be concluded that either the dominant ploidy isn't diploid (as was assumed by assigning it to be the “zero peak”), or the tumor is diluted with about 50% normal cells. The latter hypothesis is supported by the contrast between the log₂ ratio distribution of the metastatic tumor 1204, and that of the primary tumor 1202, which appears to be polyclonal. In general, the determination of whether slopes <1.0 are due to polyclonality or to mis-assignment of the diploid peak, must be made with reference to the source of the samples (e.g., samples from cell lines are unlikely to be polyclonal, whereas samples from tumors, particularly primary tumors, are quite likely to be), and/or form the results of other assays.

Applying event 1004 to the centralization curves 1202, 1204 in FIG. 12, the zero peak is identified as peak 1210 in curve 1204 and thus peak 1212 is assigned a copy number of one, peak 1214 is assigned a copy number of three peak 1216 is assigned a copy number of four and peak 1218 is assigned a copy number of five. Similarly, regarding curve 1202, peak 1220 is assigned a copy number of two, peak 1222 is assigned a copy number of one, peak 1224 is assigned a copy number of three, peak 1226 is assigned a copy number of four and peak 1228 is assigned a copy number of five. Further, the primary tumor characterized by centralization curve 1202 appears to show double peaks (peak pairs) in locations where some of the copy numbers have been assigned, which can indicate the presence of more than one cell population (clone) within the tumor. In this case, peak 1230 is assigned a copy number of two, peak 1232 is assigned a copy number of one and peak 1234 is assigned a copy number of three. In general, the assignment of peaks to copy numbers in a polyclonal sample can be difficult, but the linearity of the plot of FIG. 13 can be used to determine if the correct assignment has been made (correct sequence).

FIG. 13 shows plots of the observed ratios of these peaks (Y-axis) versus the expected ratios according to the assigned copy numbers, when carrying out event 1066. Straight lines have been fitted to the points representing the peaks for the metastasis tumor peaks (see line 1310) and for the peaks representing the clone in the primary tumor represented by the most prominent peaks (i.e., peaks 1220, 1222, 1224, 1226 and 1228, see line 1320). Thus, it can be observed that the data is substantially linear and that therefore the assigned copy numbers are most probably correct. Further, by calculating the slopes of lines 1310 and 1320, the fraction of the clone (represented by line 1310) in the primary tumor sample is calculated to be 0.35, and the fraction of the aberrant tissue in the metastatic tumor is calculated to be 0.45.

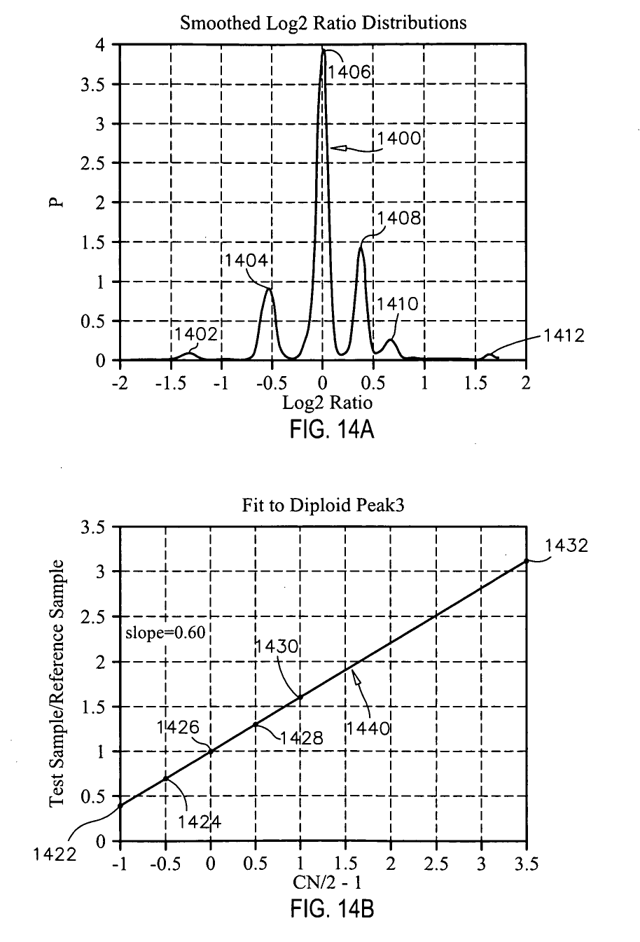

FIGS. 14A-14B illustrate an example of results from the use of the method described with regard to FIG. 10 to estimate copy numbers in the predominantly triploid cell line ht-29. The smoothed centralization curve 1400 formed as described with regard to event 1002 above is shown in FIG. 14A. After centralization, peaks 1402, 1404, 1406, 1408, 1410 and 1412 appear at log₂ ratio values of about −1.3, −0.53, 0.02, 0.39, 0.67 and 1.64, respectively. After assigning copy numbers of 1-5 and 10 and converting to ratio values and plotting as described with regard to events 1004 and 1006, it can be observed in FIG. 14B that these peaks show an excellent linear fit to assigned copy numbers of 1, 2, 3, 4, 5 and 10 to peaks 1402, 1404, 1406, 1408, 1410 and 1412, respectively, as indicated by the ratio values 1422, 1424, 1426, 1428, 1430 and 1432 in FIG. 14B, that are the converted ratio values calculated from the log₂ ratio values for peaks 1402, 1404, 1406, 1408, 1410 and 1412, respectively, and the intercept is about 1. The slope of the line 1440 is about 0.60, however, suggesting that the log₂ data of FIG. 14A have been centralized to the wrong peak, since it is unlikely that a cell line has been diluted with normal cells. A slope far from about 0.85 to about 0.95 in the plot 1440 of FIG. 14B (i.e., slope=0.60) indicates a mismatch between the assigned copy numbers of the peaks 1402, 1404, 1406, 1408, 1410 and 1412. However, since the line 1440 shows very good linearity, it can be confirmed that the sequence of the assigned copy numbers is correct.

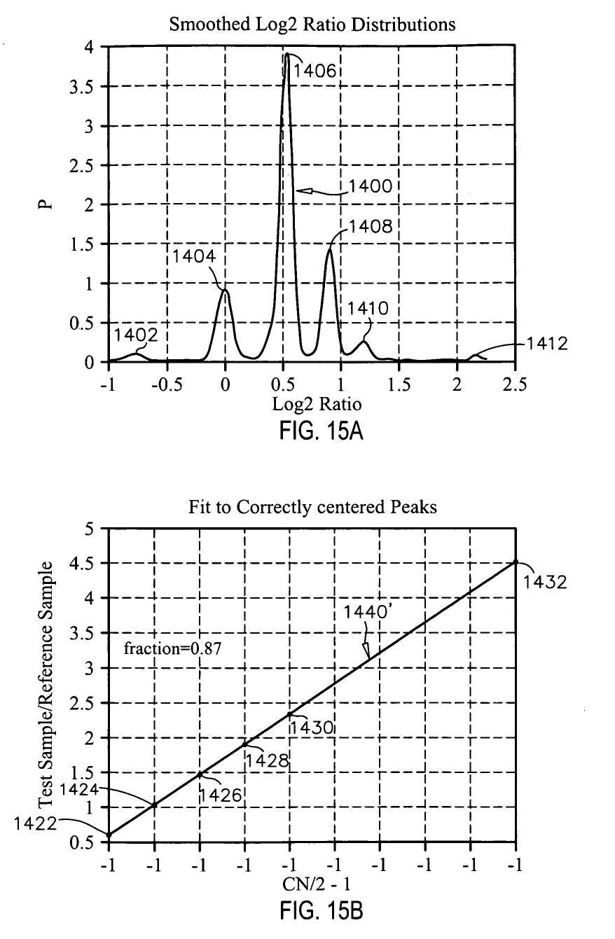

FIGS. 15A-15B illustrate centralization curve 1400′ resulting from the use of the method described with regard to FIG. 10 on the same data described above with regard to FIGS. 14A-14B, but where centralization has been performed around peak 1404 as the zero peak (as opposed to peak 1406 being the zero peak from the centralization performed with regard to FIG. 14A). Because the same sequence of copy numbers have been assigned to the peaks 1402, 1404, 1406, 1408, 1410 and 1412 (i.e., 1, 2, 3, 4, 5 and 10, respectively), the plot 1440′ in FIG. 15B also shows excellent linearity, and the y-intercept now indicates a value of one. Further, the slope is now 0.87, suggesting that the assigned copy numbers of the peaks 1402, 1404, 1406, 1408, 1410 and 1412 are most likely now correct. Although theoretically, the line 1440′ for a monoclonal sample should have a slope of 1.0, because of log ratio compression this slope is generally expected to have a slope of about 0.85 to 0.95. A slope much less than this expected value range implies either contamination of the aberrant clone with normal cells (unlikely when the test sample is from a cell line), or centralization to the wrong peak. For the ht-29 cell line, the best fit is shown in FIG. 15B, wherein the slope of line 1440′ is about 0.87.

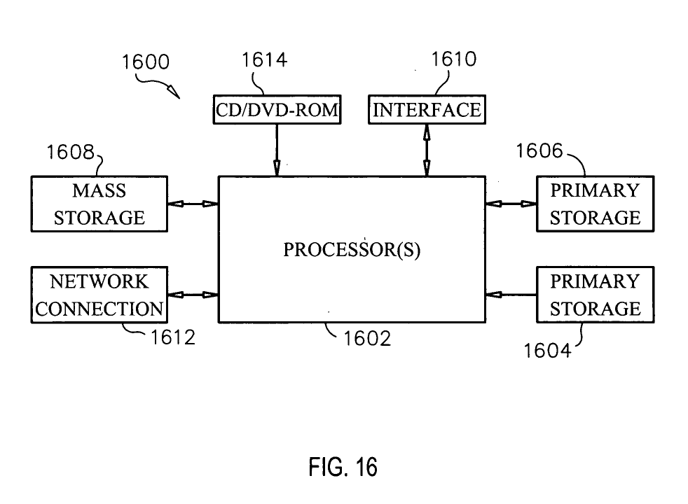

FIG. 16 is a schematic illustration of a typical computer system that may be used to perform methods described above. The computer system 1600 includes any number of processors 1602 (also referred to as central processing units, or CPUs) that are coupled to storage devices including primary storage 1606 (typically a random access memory, or RAM), primary storage 1604 (typically a read only memory, or ROM). As is well known in the art, primary storage 1604 acts to transfer data and instructions uni-directionally to the CPU and primary storage 1606 is used typically to transfer data and instructions in a bi-directional manner. Both of these primary storage devices may include any suitable computer-readable media such as those described below. A mass storage device 1608 is also coupled bi-directionally to CPU 1602 and provides additional data storage capacity and may include any of the computer-readable media described below. Mass storage device 1608 may be used to store programs, data and the like and is typically a secondary storage medium such as a hard disk that is slower than primary storage. It will be appreciated that the information retained within the mass storage device 1608, may, in appropriate cases, be incorporated in standard fashion as part of primary storage 1606 as virtual memory. A specific mass storage device such as a CD-ROM or DVD-ROM 1614 may also pass data uni-directionally to the CPU.

CPU 1602 is also coupled to an interface 1610 that includes one or more input/output devices such as such as video monitors which may be used to display outputs as well as to receive inputs in the case of an interactive user interface, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1602 optionally may be coupled to a computer or telecommunications network using a network connection as shown generally at 1612. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for plotting centralization curves, peak finding, aberration calling, etc. may be stored on mass storage device 1608 or 1614 and executed on CPU 1608 in conjunction with primary memory 1606.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). The term computer readable media is not intended to cover carrier waves. Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, sample, composition of matter, process, process step or steps, to the objective, spirit and scope of the present invention. All such modifications are intended to be within the scope of the claims appended hereto.

That which is claimed is:

1. A method of comparative genomic hybridization data analysis, said method comprising the steps of:
creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions;
identifying a peak corresponding to regions of normal copy number in said centralization curve;
centralizing the log ratio data so that the peak corresponding to regions of normal copy number is centered at a log ratio value of zero;
calculating a mathematical measurement that is a function of the width of said peak corresponding to a zero log ratio value;
calculating a tolerance value as a function of said mathematical measurement; and
outputting said tolerance value.

2. The method of claim 1, further comprising:
running an aberration calling algorithm on the log ratio data values, including the tolerance value as in input to said aberration calling algorithm for setting upper and lower threshold values; and
calling genomic regions represented by portions of the log ratio data having an average log ratio that is non-zero, but is within said upper and lower thresholds as normal regions.

3. The method of claim 1, wherein said peak corresponding to regions of normal copy number is a most prominent peak in said centralization curve.

4. The method of claim 1, wherein said peak corresponding to regions of normal copy number is selected heuristically.

5. The method of claim 1, wherein said calculating a mathematical measurement that is a function of the width of said peak corresponding to regions of normal copy number comprises fitting N Gaussian curves to N identified peaks, wherein N is a positive integer and said N peaks include said peak corresponding to regions of normal copy number; and wherein each of said Gaussian curves is defined to have the same variance.

6. The method of claim 1, wherein said centralization curve is created by a histogram.

7. The method of claim 1, wherein said centralization curve is created by
(a) plotting the log ratio data against an initial assumed location of an axis indicating a log ratio of zero;
(b) running an aberration calling algorithm on the data;
(c) tallying the fraction of the data points called in non-aberrant regions;
(d) storing the location of the axis and the fraction of non-aberrant data points as a data pair;
(e) incrementing the position of the axis indicating a log ratio of zero by a predetermined incremental value;
(f) repeating steps (b)-(e) until the axis has been incrementally moved from the initial location for log ratio of zero to both predetermined positive and negative end locations; and
(g) plotting the data pairs, with the axis position for zero log ratio values plotted along one axis and corresponding fraction values plotted along a second axis.

8. A method of comparative genomic hybridization data analysis, said method comprising the steps of:
creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions;
identifying peaks in said centralization curve;
assigning copy numbers to the identified peaks;
plotting expected ratios, based on the assigned copy numbers, of said peaks versus observed ratios of said peaks calculated from said log ratio data values;
concluding that the assigned copy numbers are correct if the plot of said expected ratios versus said observed ratios is substantially linear and the substantially linear plot is within a range of expected slope values; and
outputting at least one of the plot of expected ratios versus observed ratios, and a conclusion as to whether the plot is substantially linear.

9. The method of claim 8, wherein if the plot is not substantially linear, said method comprising reassigning a different copy number to at least one of the identified peaks to establish a new assignment of copy numbers;
repeating said plotting expected ratios versus observed ratios, using the new assignment of copy numbers;
determining whether the plot from said repeating is substantially linear; and iterating said reassigning, repeating and determining until it is determined that the plot is substantially linear.

10. The method of claim 8, wherein said expected ratios are calculated as the quantity k/2-1, where k is the assigned copy number, said method further comprising calculating the slope of the plot, wherein the slope identifies the fraction of the test sample that is aberrant.

11. The method of claim 10, further comprising outputting the value of the fraction of the test sample that is aberrant.

12. The method of claim 8, further comprising identifying multiple peak groupings indicative of a multi-clonal test sample, in the centralization curve;
wherein said assigning copy numbers to the identified peaks comprises assigning the same copy number to each peak in the same multiple peak grouping; and
wherein the assignment of copy numbers to the identified peaks within each multiple peak grouping is adjusted until said plotting results in a substantially linear plot for at least one of the clones in the multi-clonal test sample.

13. A system for analyzing comparative genomic hybridization data, said system comprising:
a processor;
a storage device in communication with the processor, said storage device storing a set of instructions that, when executed, cause the processor to cooperate with the memory device to perform the following acts:
creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions;
identifying a peak corresponding to regions of normal copy number in said centralization curve;
centralizing the log ratio data so that the peak corresponding to regions of normal copy number is centered at a log ratio value of zero;
calculating a mathematical measurement that is a function of the width of said peak corresponding to regions of normal copy number;
calculating a tolerance value as a function of said mathematical measurement; and
outputting said tolerance value.

14. The system of claim 13, wherein the storage device is programmed with a set of instructions which, when executed, cause the processor to run an aberration calling algorithm on the log ratio data values, including the tolerance value as in input to said aberration calling algorithm for setting upper and lower threshold values; and to call genomic regions represented by portions of the log ratio data having an average log ratio that is non-zero, but is within said upper and lower thresholds as normal regions.

15. A system for analyzing comparative genomic hybridization data, said system comprising:
a processor;
a storage device in communication with the processor, said storage device storing a set of instructions that, when executed, cause the processor to cooperate with the memory device to perform the following acts:
creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions;
identifying peaks in said centralization curve;
assigning copy numbers to the identified peaks;
plotting expected ratios, based on the assigned copy numbers, of said peaks versus observed ratios of said peaks calculated from said log ratio data values;
concluding that the assigned copy numbers are correct if the plot of said expected ratios versus said observed ratios is substantially linear; and
outputting at least one of the plot of expected ratios versus observed ratios, and a conclusion as to whether the plot is substantially linear.

16. The system of claim 15, wherein the storage device is programmed with a set of instructions which, are executed if the plot is not substantially linear, wherein the instructions when executed, cause the processor to:
reassign a different copy number to at least one of the identified peaks to establish a new assignment of copy numbers;
repeat said plotting expected ratios using the new assignment of copy numbers;
determine whether the plot from said repeat is substantially linear; and iterate said reassign, repeat and determine steps until it is determined that the plot is substantially linear.

17. The system of claim 15, wherein the storage device is programmed with a set of instructions which, when executed, cause the processor to calculate said expected ratios as the quantity k/2-1, where k is the assigned copy number; and calculate the slope of the plot, wherein the slope identifies the fraction of the test sample that is aberrant.

18. The system of claim 15, wherein the storage device is programmed with a set of instructions which, when executed, cause the processor to identify multiple peak groupings indicative of a multi-clonal test sample, in the centralization curve, wherein said assigning copy numbers to the identified peaks comprises assigning the same copy number to each peak in the same multiple peak grouping, wherein the assignment of copy numbers to the identified peaks within each multiple peak grouping is adjusted until said plotting results in a substantially linear plot for at least one of the clones in the multi-clonal test sample.

19. A computer readable medium carrying one or more sequences of instructions for analysis of comparative genomic hybridization data, wherein execution of one or more sequences of instructions by one or more processors causes the one or more processors to perform the steps of:
creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions;
identifying a peak corresponding to regions of normal copy number in said centralization curve;
centralizing the log ratio data so that the peak corresponding to regions of normal copy number is centered at a log ratio value of zero;
calculating a mathematical measurement that is a function of the width of said peak corresponding to regions of normal copy number;
calculating a tolerance value as a function of said mathematical measurement; and
outputting said tolerance value.

20. A computer readable medium carrying one or more sequences of instructions for analysis of comparative genomic hybridization data, wherein execution of one or more sequences of instructions by one or more processors causes the one or more processors to perform the steps of:
creating a centralization curve from log ratio data values for DNA copy numbers of a genome of a test sample relative to a genome of a reference sample, wherein the reference sample has a known ploidy, and the test sample has a same copy number as the reference sample in normal, non-aberrant genomic regions;
identifying peaks in said centralization curve;
assigning copy numbers to the identified peaks;
plotting expected ratios, based on the assigned copy numbers, of said peaks versus observed ratios of said peaks calculated from said log ratio data values;
concluding that the assigned copy numbers are correct if the plot of said expected ratios versus said observed ratios is substantially linear; and
outputting at least one of the plot of expected ratios versus observed ratios, and a conclusion as to whether the plot is substantially linear.
